(19) United States
(12) Patent Application Publication
(10) Pub. No.: US 2012/0143176 A1
(43) Pub. Date: Jun. 7, 2012

(54) LASER SYSTEMS AND METHODS FOR VAPORIZATION OF PROSTATE AND OTHER TISSUE

(75) Inventors: Andrew Ryan, Atherton, CA (US); Ramsey Shanbaky, San Francisco, CA (US)

(73) Assignee: Metalase, Inc., San Jose, CA (US)

(21) Appl. No.: 13/309,033

(22) Filed: Dec. 1, 2011

Related U.S. Application Data
(63) Continuation-in-part of application No. 13/051,891, filed on Mar. 18, 2011.
(60) Provisional application No. 61/315,338, filed on Mar. 18, 2010.

(51) Int. Cl. A61B 18/20

(52) U.S. Cl. 606/3

(57) ABSTRACT

The present invention provides systems, devices, and methods to vaporize prostatic tissue having oxygenated hemoglobin. The systems, devices, and methods include a laser module configured to emit light near an absorption peak of the oxygenated hemoglobin and a delivery catheter that is optically coupled to the laser module and configured to deliver the light from the laser module to the prostatic tissue.

### CROSS-REFERENCES TO RELATED APPLICATIONS

The present application is a continuation in part of U.S. patent application Ser. No. 13/051,891, filed Mar. 18, 2011, which claims priority from Provisional U.S. Patent Application Ser. No. 61/315,338, filed Mar. 18, 2010, the full disclosures of which are incorporated herein by reference.

### BACKGROUND

Prior systems and methods to treat tissue pathology with laser systems are less than ideal. In at least some instances, the laser systems can be complex and less effective than ideal. Also, at least some laser systems provide light energy that is less than ideal for the treatment of at least some tissues, for example hemoglobin. Although electrodes can be used to treat pathological tissue such as dysplasia, the heat energy generated with electrodes may not be tissue specific, such that in at least some instances the treatment can damage healthy tissue.

### BRIEF SUMMARY

Embodiments of the invention are directed to the treatment of tissue with lasers, in particular the vaporization of tissue comprising hemoglobin. Although specific reference is made to the vaporization of tissue for the treatment of Benign Prostatic Hyperplasia (BPH), various embodiments can be used to vaporize many tissues, for example tissue comprising hemoglobin.

Embodiments of the present invention provide improved laser systems and methods for vaporization of tissue such as prostatic tissue, for example in patients with Benign Prostatic Hyperplasia (BPH). The systems and methods described here can also be used in many applications where rapid hemostatic vaporization of tissue is desirable, for example, in BPH tissue comprising hemoglobin.

In some embodiments, a medical laser system is configured that produces light with an optical wavelength to treat tissue having oxygenated hemoglobin. In some embodiments, the laser system can treat prostatic tissue. For example, in performing prostatectomy to treat BPH, nonmalignant neoplasm of the prostatic epithelial gland can be ablated by rapid vaporization of cellular water and, in a subset of the embodiments, a less than 1-mm ring of coagulated tissue will be left behind to inhibit bleeding. The laser beam energy and emitted wavelength can be particularly well-suited for treatment of BPH. For example, the wavelength of light can be within a range from about 390 nm to about 430 nm, which is near the peak of absorption of oxygenated hemoglobin. The prostatic tissue can be ablated substantially and the hemostatic coagulation layer thickness can be substantially reduced (compared to existing treatments) such that post-operative dysuria is reduced.

In some embodiments, the medical laser system may comprise a laser diode module having one or more laser diodes configured to emit light energy near an absorption peak of oxygenated hemoglobin, for example near the absorption peak of 416 nm. The one or more laser diodes can be coupled to a delivery catheter having a light delivery channel, such that light flux energy within a range from about 1 kW/cm² to 10 kW/cm² can be delivered to tissue such that the prostatic tissue can be ablated and underlying tissue can be coagulated. The treated tissue may comprise a first portion removed with ablation and a second coagulated portion. The one or more laser diodes may comprise a plurality of laser diodes coupled to a plurality of optical fibers, and the light energy from the plurality of optical fibers can be combined so as to provide a smoothed laser beam energy profile distribution. The plurality of optical fibers can be coupled to a mono-mode or multi-mode optical delivery catheter, and the delivery catheter can be inserted into the patient. The distal end of the an optical fiber inserted into the patient can be scanned with movement of the distal end so as to treat an area of the patient substantially larger than the distal end of the optical fiber. The various embodiments described in the summary and in the rest of the written description are not meant to be limiting. The scope of the invention is not defined by any part of the written description, but rather is defined solely by the claims.

### BRIEF DESCRIPTION OF THE DRAWINGS

FIG. 1A shows a diagram of a laser prostatectomy procedure using a cystoscope in accordance with some embodiments of the present invention.

FIG. 1B shows the insertion of a prostatic laser delivery catheter into the working channel of a cystoscope for use in a laser prostatectomy procedure in accordance with some embodiments of the present invention.

FIG. 2A shows an image of a prostate viewed through a cystoscope before a prostatectomy procedure and FIG. 2B shows an image of a prostate viewed through a cystoscope after a prostatectomy procedure.

FIG. 3 shows an absorption spectrum of hemoglobin, an absorption spectrum of prostate tissue, and a scattering spectrum of prostate tissue in accordance with embodiments of the present invention.

FIG. 4 shows heat flux versus depth and wavelength.

FIG. 5 shows a laser system schematic in accordance with some embodiments of the present invention.

FIG. 6 shows a cross section of a delivery catheter in accordance with embodiments of the present invention.

FIGS. 7A-7C show the delivery catheter placed in the prostate during treatment in accordance with embodiments of the present invention.

FIG. 8A shows a coupler in an off-position and FIG. 8B shows a coupler in an on-position in accordance with embodiments of the present invention.

FIG. 8C shows an aim diode for use in a coupler in accordance with embodiments of the present invention.

### DETAILED DESCRIPTION OF THE INVENTION

Embodiments of the invention can exploit the absorption peaks of molecules such as hemoglobin for tissue ablation purposes. Laser ablation at the absorption peaks of molecules in the tissue allows for more efficient ablation.

As used herein, a tissue treatment region encompasses an ablation layer and a coagulation layer.

Embodiments of the invention can be used for laser treatment of abnormal tissue in the prostate; for example, benign prostatic hyperplasia (BPH). The system and method described herein can also be used in many applications where the treatment region comprises shallow surface layers where minimal damage to the tissue beneath is desirable. The treatment region may include any tissue comprising hemoglobin.

BPH is a nonmalignant neoplasm of the prostatic epithelial and stromal tissue that causes enlargement of the prostate gland and may result in bladder outlet obstruction. BPH is common among older men in the United States and worldwide. Most frequently, obstructive symptoms of BPH occur between the ages of 65 and 70 years. Approximately 65% of men in this age-group have prostatic enlargement from hyperplasia. Embodiments of the present invention can be used for the treatment of BPH.

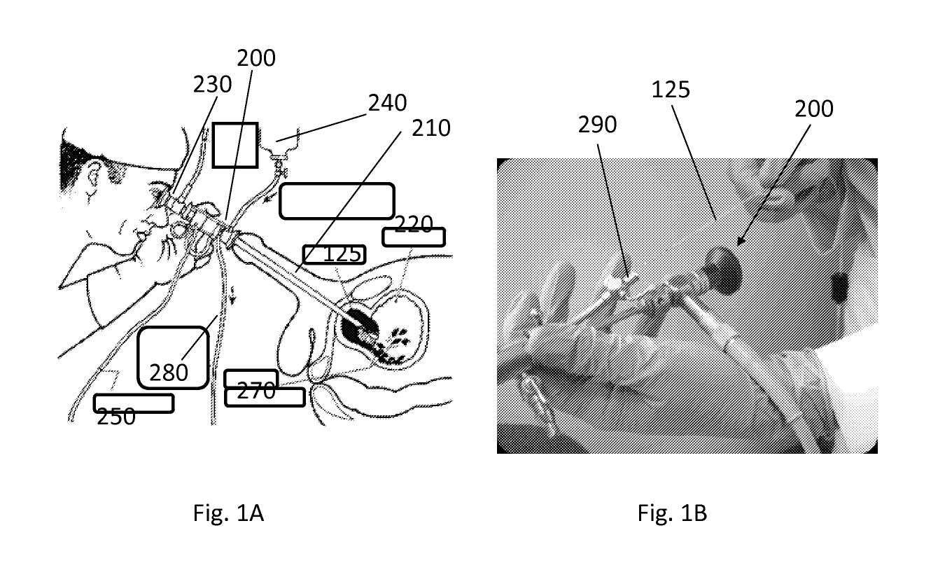

FIG. 1A shows laser transurethral resection of the prostate (TURP) treatment of BPH. A cystoscope 200 is inserted through the urethra 210 of the patient. The surgeon positions the distal end of the delivery catheter near the target prostatic tissue. The surgeon views the interior or the prostate 220 through the cystoscope eyepiece 230. Irrigant 240, typically saline solution, can flow into and out of the patient through channels in the cystoscope. A light source 250 can be used to illuminate the interior of the prostate. A delivery catheter 125 delivering laser energy can be passed through a working channel of the cystoscope. The delivery catheter can be used to ablate prostate tissue. Laser ablation within the prostate produces small pieces of debris 270 which flow out with the irrigant through the cystoscope exit channel 280. The surgeon or other medical professional can use the prostatic treatment system having a distal end to ablate at least a portion of the prostatic tissue by vaporizing at least a portion of the cellular water. In some embodiments, the laser can emit light with a wavelength between 390 nm and 430 nm.

In some embodiments, the laser can operate with a power output between 6 W and 60 W. In other embodiments the power output can be between 15 W and 25 W. In some embodiments, delivery catheter 125 can deliver a laser beam on an object with a spot size between 0.5 mm and 2.5 mm. In some embodiments, the laser delivery channel can have a diameter greater than 0.05 mm and less than 1.5 mm. In other embodiments, the laser delivery channel can have a diameter greater than 0.35 mm and less than 0.65 mm. In some embodiments, the laser can emit light with a flux less than or equal to 10 kW/cm² and/or greater than 1 kW/cm² In some embodiments, the laser can emit light with a pulse duration between 80 ms and 160 ms. In some embodiments, the laser can emit light with a pulse repetition rate between 4 Hz and 12 Hz.

FIG. 1B is a photo of the surgeon inserting laser delivery catheter 125 into the working channel 290 of the cystoscope 200. The cystoscope may be a rigid cystoscope or a flexible cystoscope. In various embodiments, prostatic delivery catheter 125 has an outer diameter between 0.4 mm and 0.6 mm, and is delivered through a flexible cystoscope. The delivery catheter 125 may be a delivery fiber. In some embodiments, laser delivery catheter can be inserted directly into the patient's urethra 210.

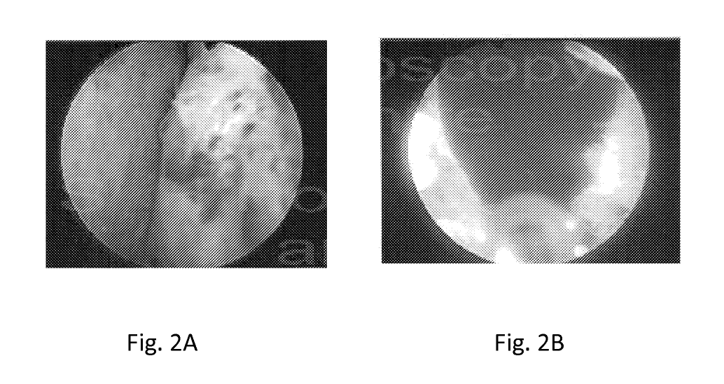

FIG. 2 shows views of a prostate through a cystoscope. In FIG. 2A, the prostate, which is enlarged due to BPH, is obstructing the flow of urine. FIG. 2B shows the prostate after treatment has opened up a channel to enable flow.

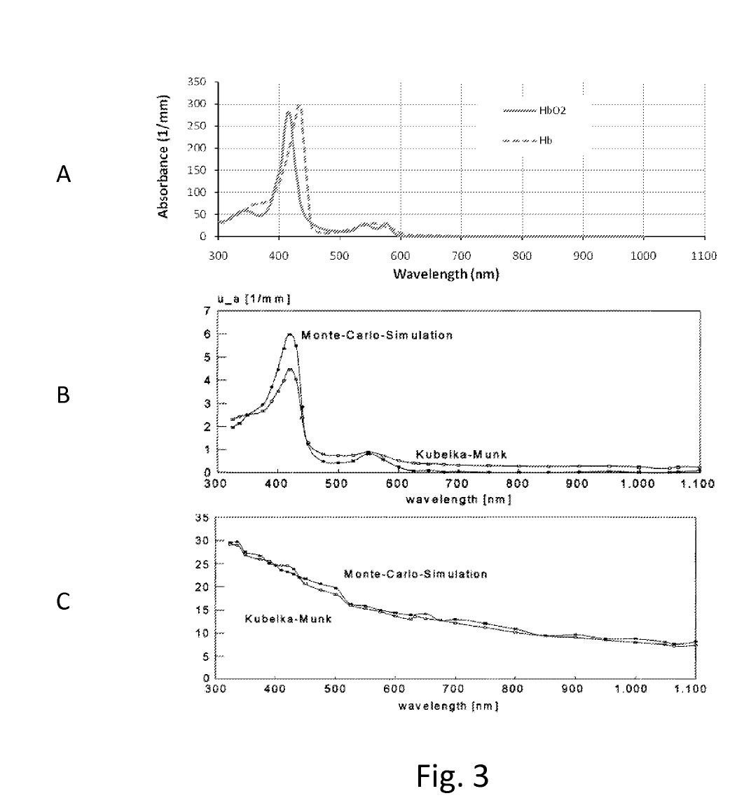

The presence of hemoglobin in the prostatic neoplasm allows the system to use light energy to treat the tissue by exploiting the high absorption of hemoglobin. FIG. 3A shows the hemoglobin absorption spectra for oxygenated hemoglobin (HbO₂) and non-oxygenated hemoglobin (Hb). The average peak absorption wavelength for hemoglobin is about 420 nm, although the peak absorption wavelength for non-oxygenated hemoglobin (Hb) is about 433 nm while the peak absorption wavelength for oxygenated hemoglobin (HbO₂) is 416 nm. FIG. 3B shows the absorption spectrum of prostate tissue as measured by Roggan et al. The position and relative height of the peaks is similar to hemoglobin. FIG. 3C shows the scattering spectrum of prostate tissue as measured by Roggan et al. The wavelength dependence of scattering is much weaker than that of absorption and is substantially unaffected by the presence of hemoglobin.

In many embodiments, a medical laser system can be configured to emit light energy from diode lasers of a diode laser module. The light energy emitted from the laser diodes of the laser diode module can be used to treat tissue near the 420 nm absorption peak of hemoglobin. For example, 405 nm light energy is near the 420 nm peak and even nearer to the 416 nm absorption peak of oxygenated hemoglobin, such that tissue comprising hemoglobin can be selectively treated with the light energy emitted from the laser diodes. The system can be configured to pulse the laser diodes to generate a pulsed laser beam to treat or ablate the tissue. The auto absorbance of the tissue comprising hemoglobin can allow a thin layer of a first pathological tissue comprising hemoglobin to be treated. The system may comprise an adjustable laser beam pulse intensity and duration to selectively treat the targeted tissue.

The treatment of tissue as described herein may comprise deposition of energy sufficient to substantially alter the tissue so as to achieve a desired therapeutic benefit. For example, the treatment may comprise heating the tissue so as to coagulate the tissue, and in many embodiments the tissue can be ablated. Ablation of tissue as described herein encompasses removal of the tissue, for example with heating of the tissue to 100° C. so that some water in the tissue vaporizes.

In the visible light region, the absorption of the hemoglobin molecule can be exploited. Embodiments as described herein can exploit the largest hemoglobin absorption features near 420 nm. The wavelengths used can be within +/−50 nm of the peak, within +/−40 nm of the peak, within +/−30 nm of the peak, within +/−20 nm of the peak, within +/−10 nm of the peak, or within +/−5 nm of the peak. For example, the wavelength emitted can be within +/−20 nm of the peak and may comprise a wavelength of about 405 nm from a gallium-nitride (GaN) laser diode. Several of these GaN diode laser devices can be combined, for example ganged together, and used to produce higher power modules. The maximum power available from a commercially available single GaN laser diode operating near 405 nm is currently specified as 700 mW, although there have been demonstration devices of 3,000 mW at 450 nm. The fiber-coupled power available around 405 nm from the combined fibers in a 20-emitter module can be 10 W. In this module, each of the emitters can be polarization can be combined into an independent 0.125 mm (numerical aperture 0.2) diameter fiber, which are then collected together to fill a 0.650 mm aperture.

The physics of the laser-tissue interaction can be combined in accordance with the embodiments as described herein. The laser beam intensity is affected by both absorption and scattering as it propagates through the tissue. In the first order, the intensity of the electromagnetic radiation as a function of depth in tissue is governed by Lambert's Law for turbid media, as follows:

I(z)=I₀ exp [−(α+αₛ)z] (EQ. 1)

Where z is the depth in the tissue, I₀ is the optical intensity at z=0, α is the absorbance of the medium and αₛ is the scattering coefficient of the medium. The wavelength-dependence of the tissue response enters through the wavelength dependence of the absorbance and the scattering coefficient. The optical penetration depth, Lₒ, is determined from the inverse of these two parameters, as in the following equation:

Lₒ=1/(α+αₛ) (EQ. 2)

Lₒ corresponds to the depth in the tissue at which the optical intensity is reduced to 1/e of its value at the surface.

A more sophisticated analysis incorporating the effect of the absorption of scattered light, called the Diffusion Approximation, separates the laser intensity into coherent, I_{c}, and diffuse, I_{d} components.

[symbol]

I=I_{c}+I_{d}=I_{c0} exp [−(α+αₛ)z]+I_{d0} exp−α_{d}z) (EQ. 3)

This simplified expression suppresses the z-dependence of I_{d0} but captures the significance of the diffuse component. The effective attenuation of the diffuse component, α_{d}, is:

[symbol]

α_{d}={square root over (3(α^{g}+ααₛ(1−g)))} (EQ. 4)

The g parameter accounts for the directionality of the scattering where g=1 denotes purely forward scattering, g=0 denotes purely isotropic scattering and g=−1 denotes purely backward scattering. For tissue using visible wavelengths g≈0.90-0.95.

Heat is generated inside the tissue during laser exposure by means of a two step process. First, photon energy is absorbed by the molecules that comprise the tissue. These molecules then decay from their excited state via inelastic collisions with particles in the surrounding medium. Therefore, the temperature rise originates microscopically from the transfer of photon energy to kinetic energy. In the first order, the heat flux in the medium is given by:

S(z,λ)=α1(z,λ)=α1₀ exp [−(α+αₛ)z] (EQ. 5)

The wavelength dependence of the tissue response enters through the wavelength dependence of the absorbance, α, and scattering, αₛ. Generally, while both parameters may have roughly equal influence on the evolution of intensity, the absorption parameter dominates the generation of heat.

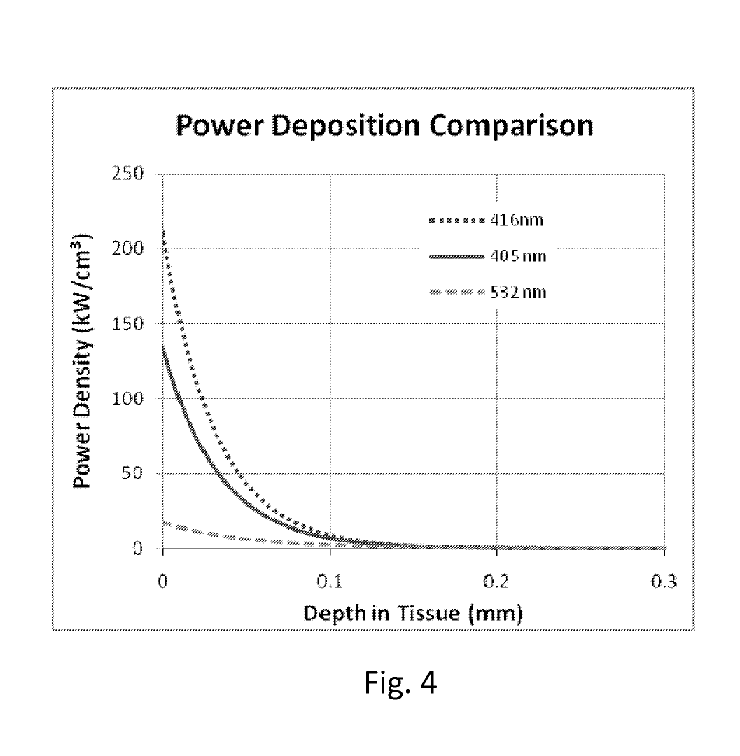

The relationship between these parameters is illustrated in FIG. 4 by comparing the heat flux provided by beams at different wavelengths, in particular 416 nm, 405 nm, and 532 nm for a 10 W, 650 μm diameter beam. For a 416 nm beam, the absorbance of oxygenated hemoglobin is approximately 2795 cm⁻¹ which is roughly 11.9 times larger than the 235 cm⁻¹ absorbance of oxygenated hemoglobin at 532 nm. For a 405 nm beam, the absorbance of oxygenated hemoglobin is approximately 1775 cm⁻¹ which is roughly 7.5 times larger than the 235 cm⁻¹ absorbance of oxygenated hemoglobin at 532 nm. For tissue comprised of 2.5% oxyhemoglobin these numbers are 69.9 cm⁻¹, 44.7 cm⁻¹, and 5.9 cm⁻¹ for the 416 nm absorption, the 405 nm absorption, and the 532 nm absorption, respectively. The corresponding scattering coefficients measured in tissue are 245 cm^{31 1}, 250 cm⁻¹, and 190 cm⁻for the scattering at 416 nm, 405 nm, and 532 nm respectively. For a 20 W beam that uniformly fills a 650 μm aperture, I₀=6.02 kW/cm². Using these numbers, the heat flux can be graphed as a function of depth in tissue for the first order approximation (see FIG. 4). At each of the three wavelengths nearly all of the energy is deposited in the first 100 μm of tissue. The difference is that the much larger absorption at 405 nm and the 416 nm corresponds to significantly more energy being deposited by the 405 nm and the 416 nm beams. A Diffusion Approximation treatment would be qualitatively similar but show more energy deposition by the 532-nm beam at greater depths.

One method of using the first order approximation to calculate the conditions required for vaporization is to calculate the conditions required to vaporize the water in the tissue. The vaporization of water in tissue occurs in two steps. First, the temperature of the tissue being vaporized is heated to 100° C. Assuming that the specific heat of tissue is approximately equal to the specific heat of water, that the tissue begins at 37° C., and that the tissue is 80% water by weight, the heat required to raise a mass of tissue, m, to 100° C. can be calculated as Q₁ with the following equation:

Q₁=mcΔT=m(0.8)(4.3 J/g)(63 C)=m×217 J/g (EQ. 6)

Next, the heat required to vaporize that same mass of tissue can be calculated. The energy required to vaporize the tissue can be the energy require to vaporize the water within the tissue, thus:

Q₂=mQᵥₐₚ=m(0.8)(2253 J/g)=m×1802 J/g (EQ. 7)

Given the assumption that the density of the tissue is approximately the same as that of water, 1 g of tissue would have a volume of approximately 1 cubic centimeter. The heat of vaporization per unit volume can then be calculated by the following equation:

Qₜₒₜₐₗ/V=(Q₁+Q₂)/V=2019 J/cm³ (EQ. 8)

Given the above definitions, the heat flux at a depth of one optical penetration depth, L_{o,} is considered using the following equation:

S(Lₒ,λ)=αI₀ exp [−(α+αₛ)Lₒ]=αI₀/e (EQ. 9)

Thus, for the 416 nm beam (20 W at 416 nm emitted from 650 μm core as described above), the volumetric flux is 145 kW/cm³ at the optical penetration depth of 33.9 μm. For the 405 nm beam (20 W at 405 nm emitted from 650 μm core as described above), the volumetric flux is 99.0 kW/cm³ at the optical penetration depth of 33.9 μm. At that same depth, the flux from a 532 nm beam of the same power and size would be only 18.2 kW/cm³ due largely to the difference in absorbance. At these respective fluxes, the 416 nm beam would take 14 milliseconds (14×10⁻³ s) to vaporize the tissue to its optical penetration depth and the 405 nm beam would take slightly more than 20 milliseconds (20×10⁻³ s) to vaporize the tissue to that depth, while the 532 nm beam would require more than 110 ms to vaporize to that same depth. More generally, the 416 nm beam with a 650 μm diameter can vaporize a 33.9 μm layer with just 279 mJ of energy and the 405 nm beam with a 650 μm diameter can vaporize the 33.9 μm layer with 408 mJ of energy, whereas a 532 nm beam with the same cross section requires 2210 mJ. In the latter case, the extra energy (1931 mJ of energy as compared to the 416 nm beam and 1802 mJ of energy as compared to the 405 nm beam) continues to penetrate and damage the tissue beneath the vaporization layer. Thus, the higher absorption provided by the 416 nm and the 405 nm beams allows for vaporization of tissue while reducing thermal damage to the tissue beneath. Moreover, vaporization of a given amount of tissue with the 405 nm beam requires less total energy than would the 532 nm beam. It should be noted that this calculation has been simplified by neglecting the effect of the absorption of scattered light. As before, the details will change but the basic conclusions will remain the same as these higher order effects are included.

The above example is meant to be illustrative. The same physics holds true for lower or higher power beams with larger or smaller diameters. Generally, a 405 nm light will vaporize a given tissue layer in less time than would a 532 nm light. Additionally, more excess energy will be delivered to lateral and deeper tissue by the more weakly absorbed wavelength, thus producing more necrotic tissue which increases the likelihood of dysuria. The above calculation has also been simplified by neglecting the flow of heat away from the irradiated region. Heat flow affects the total amount of energy required to achieve vaporization, but the higher-absorbed wavelength, in this example the 405 nm beam, will typically require less energy to vaporize a layer of a given thickness than would a lower-absorbed wavelength. The same physics also determines the absorption and vaporization interaction at other wavelengths. In particular, the least amount of energy is required for a beam at approximately 420 nm for hemoglobin, approximately 433 nm for non-oxygenated hemoglobin, and approximately 416 nm for oxygenated hemoglobin.

It should further be noted that in laser treatment of tissue, coagulation and necrosis typically occur for tissue heated above 60 degrees Celsius. Using the same methodology as noted above, a 23 degree temperature rise would be required and the flux required to achieve coagulation can be calculated by the following equation:

Q₁=mcΔT=m(0.8)(4.3 J/g)(23 C)=m×79.1 J/g (EQ. 10)

In many applications, limiting the depth of tissue in which coagulation is produced is desirable, however, a more detailed thermal model may be required to make effective predictions about the conditions under which coagulation occurs at depths significantly greater than the optical penetration depth. Such a detailed thermal model may account for the lateral diffusion of heat as well as the diffusion of heat during the pulse.

It is desirable to minimize the coagulation layer in treating BPH patients as it may lead to negative side effects such as dysuria. However, one benefit of the coagulation layer is that it provides hemostasis, which prevents bleeding following treatment. Lasers that penetrate even less than 405 nm, e.g. a diode laser operating at the 416 nm absorption peak, might not produce enough of a coagulation layer to provide hemostasis. In one method, the surgeon uses parameters that leave a hemostatic coagulation layer. For example, the surgeon might choose a light wavelength that sufficiently penetrates the tissue to form a hemostatic coagulation layer.

Another benefit of the lower power requirement of the 405 nm light is an increased allowable optical fiber bend radius in the optical fiber. Fiber bend radius is the radius at which an optical fiber can be safely bent without breaking. As the power in the fiber increases, the allowable fiber bend radius increases as well. Additionally, at shorter wavelengths, the spatial extent of the evanescent fields of the light inside the fiber is smaller resulting in less light energy being lost upon bending through the fiber. The combination of low power and short wavelengths allows for delivery catheters having narrow diameters such as 500 μm or less such as are optimal for use in a flexible cystoscope.

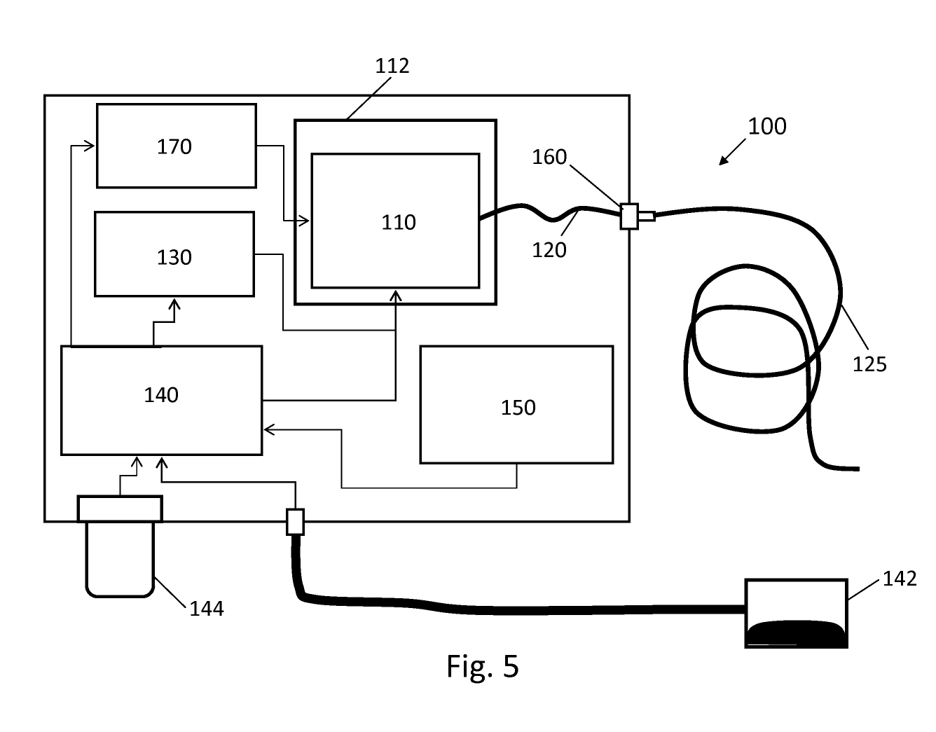

FIG. 5 shows a schematic of system 100 according to some embodiments of the invention. In this embodiment, system 100 comprises a laser diode module 110 disposed in a heat sink 112 such that the laser diode module 110 is thermally coupled to the heat sink 112. The laser diode module 110 emits light energy to fiber cable 120, which is optically coupled to a delivery catheter 125 for delivering the light energy to the tissue treatment region. Optionally, the delivery catheter 125 is optically coupled to fiber cable 120 by coupler 160. In this embodiment, the delivery catheter 125 is configured to be inserted into a cystoscope for further insertion through the urethra of a patent to treat abnormal tissues in the prostate, such as BPH. The system 100 further comprises a DC power supply 170, a pulse generator 130, a control module 140, and a user interface 150, such as a touch screen. The control module 140 may be configured to receive user input from a control, such as a footswitch 142 for turning the laser treatment on and off; from a user interface 150 for entering treatment parameters; and from an authorization input such as a security card 144 inserted into a card reader.

In an exemplary embodiment, system 100 comprises a laser diode module 110. Laser diode module 110 includes at least one laser diode to emit light energy, but may include multiple diode lasers. In one embodiment, the laser diode module 110 is a diode laser bar containing a one-dimensional array of light emitters. In select embodiments, the laser diode module comprises a plurality of diode lasers coupled to a plurality of optical fibers, and each diode laser can be coupled to an optical fiber such that the laser diodes are each individually coupled to at least one of the optical fibers. The plurality of diode lasers many include any number of diodes lasers. For example, the plurality of diode lasers may include 10, 38, or 900 diode lasers. The one or more optical fibers can be collected together so as to comprise fiber cable 120. The system may comprise a control module 140 configured to send signals such as on or off signals and power level signals to the laser diode module 110. The signals can be received by the control module 140 from a footswitch 142 depressed by the user when the user wants to initiate treatment. A temperature sensor may monitor the temperature of the laser module. The system may comprise a user interface 150 wherein the user enters pulse and power parameters that determine the treatment. The system may comprises a pulse generator 130 coupled to the control module 140, such that the pulse generator 130 receives signals from control module 140 so as to determine the duration, frequency and amplitude of the pulses sent to the laser diode module 110 to provide the pulse energy amount and duration of the laser beam. The system may comprise a power supply 170, which provides energy to drive the laser diode module 110 and the other electrical system components. The system 100 may further comprise a user interface 150 wherein data from the user is entered and system data is displayed. The laser diode module 110, pulse generator 130, DC power supply 170, control module 140, and user interface 150 can be housed within a chassis or housing.

In many embodiments, the fiber cable 120 can be connected to the chassis wall via a fiber connector or coupler 160, the fiber connector can provide efficient coupling of light energy from the fiber cable 120 to the prostatic delivery catheter 125. The chassis wall may include a connection port for the footswitch 142, and a connection where AC power may be delivered to the system. The chassis wall may include a USB port where a computer or a diagnostic tool may be connected to access system data. Optionally, the chassis wall may include a security-card reading port configured to receive security card 144 to ensure that only adequately designed delivery catheters 125 are used with the system.

Laser Diode Module

In an exemplary embodiment, system 100 comprises at least one laser diode module 110 to emit light energy, preferably at a wavelength suitable for absorption by an abnormal tissue. For example, to treat BPH, the at least one laser diode module 110 may emit light energy having a wavelength within a range from about 360 nm to about 450 nm, 400 nm to 430 nm, or 400 nm to 410 nm. In one embodiment, the laser is configured to emit light with a wavelength between 415 nm and 425 nm. In another embodiment, the laser is configured to emit light with a wavelength between 390 nm and 430 nm. In yet another embodiment, the laser is configured to emit light with a wavelength between 400 nm and 425 nm. Typically, the output power of the system delivered to the tissue is at least about 10 W and less than 60 W, although other output powers are within the scope of the invention. For example, the maximum power output of various embodiments ranges from 6 W to 60 W, or even from 6 W to 30 W, or even from 0.1 W to 10 W, or even 0.01 W to 1,000 W. The laser diode module 110 may comprise one laser diode optically coupled to an optical fiber or to a bundle of fibers, or may comprise multiple diodes coupled to an optical fiber or a bundle of fibers for directing the light energy to the treatment site. In an exemplary embodiment, the optical light flux energy exiting the at least one optical fiber corresponds to an optical light flux energy on a surface of the tissue within a range from about 1,000 W/cm² to about 9,000 W/cm². In some embodiments, the optical light flux energy or the optical light flux energy exiting the at least one fiber is within the range from 1,000 W/cm² to 9,000 W/cm² when a distal end of the at least one fiber is placed within 1 mm of the surface of the tissue. In various embodiments, the system delivers the light flux energy while the distal end of the system is located 1.0 mm to 2.0 mm, 1.5 mm to 2.5 mm, 1.0 mm to 3.0 mm, or 3.0 mm to 5.0 mm from the portion of the prostatic tissue being ablated. In another embodiment, the system is used while the distal end of the at least one fiber is placed within about 2 mm from the tissue targeted for ablation. In yet another embodiment, the system is used while the distal end of the at least one fiber is located 5 mm to 0.1 mm from the tissue targeted for ablation. In one embodiment, the system delivers a light flux energy within a range from 500 W/cm² to 10,000 W/cm² to the prostatic tissue. In another embodiment, the system delivers a light flux energy within a range from 500 W/cm² to 5,000 W/cm² to the prostatic tissue. In another embodiment, the system delivers a light flux energy within a range from 400 W/cm² to 3,000 W/cm² to the prostatic tissue. In yet another embodiment, the system is configured to emit a maximum flux of at least 1 kW/cm² and is configured to vaporize the prostatic tissue by emitting a fluence of less than 5 kW/cm². In another embodiment, the system is configured to vaporize the prostatic tissue by emitting a fluence of less than 10 kW/cm². In at least one embodiment, pulsing the laser comprises emitting a fluence of less than 10 kW/cm² to the target prostatic tissue. In another embodiment, pulsing the laser comprises emitting light with a wavelength between 390 nm and 430 nm, or between 400 nm and 410 nm, or between 415 nm and 425 nm.

In one embodiment, laser diode module 110 comprises the NUV101E slot module sold by Nichia Corporation. The NUV101E includes 20 laser emitters optically coupled to individual fibers which are then combined at an optical connector into a 650 μm diameter fiber cable 120 with a numerical aperture of 0.2. The NUV101E emits light in the wavelength region between 400 and 410 nm; requires a 24 V and 4 A regulated DC power supply; and voltage pulses up to 3.5 V. The typical maximum power output is at least about 10 W. The NUV101E returns a signal proportional to the submount temperature of the diode lasers. The NUV101E also has LEDs that indicate the condition of the module: An illuminated green LED means the module has DC power and an illuminated red LED means a fault condition is occurring (e.g., high temperature or a voltage signal that is too high). If there is no illuminated LED, then the module is not receiving power. In some embodiments, the operating temperature of the laser diode module 110 is between 20° C. and 30° C. In another embodiment, the laser diode module 110 may include two NUV101E slot modules and may use polarization beam combining to produce a 20 W beam.

In one embodiment, the system includes a prostatic delivery catheter with a core diameter between 0.4 mm and 0.6 mm, and the system is configured to emit a beam wherein the beam's power is between 15 W and 25 W. In one embodiment, the system is configured to vaporize prostatic tissue with a power output between 15 W and 25 W. In one embodiment, the core diameter is the diameter of the light delivery channel 127 inside the delivery catheter 125. The beam may have a spot diameter between 0.9 mm and 1.3 mm; the beam may have a fluence between 1.8 kW/cm² and 2.4 kW/cm²; and the beam may have a numerical aperture between 0.1 and 0.2. In another embodiment, the beam has a spot diameter between 1.86 mm and 2.06 mm; the beam has a fluence between 0.56 kW/cm² and 0.76 kW/cm²; and the beam has a numerical aperture between 0.25 and 0.45. In various embodiments, one system has a spot diameter between 0.5 mm and 2.5 mm; another system has a spot diameter between 1 mm and 2 mm; another system has a spot diameter of approximately 1.1 mm; and another system has a spot diameter of approximately 1.96 mm.

In another embodiment, the laser diode module 110 includes a plurality of individually packaged diode lasers mounted on peltier coolers. In this embodiment, each of the diode lasers are coupled to an optical fiber. Individual diode lasers are typically packaged in TO-5 cans, TO-9 cans, or on a C-mount. The light coupling may be further assisted with the use of cylindrical lenses. Using such techniques, coupling efficiency between a given laser diode and its associated optical fiber may surpass 80%. In one embodiment, the laser diode module 110 comprises NDV7112-E individual laser diodes sold by Nichia Corporation. These devices have maximum optical power of 700 mW and an emission wavelength between 400 nm and 405 nm. The operating current per laser diode is between 450 mA and 650 mA with a slope efficiency between 1.0 W/A and 2.0 W/A. The operating voltage of the diodes is between 3.8 V and 4.6 V, and the operating temperature is between 20° C. and 30° C. In one embodiment, laser diode module 110 comprises 19 individually packaged and coupled diode lasers optically coupled to the fiber cable 120. The diode lasers are mounted such that they may receive voltage pulses simultaneously and with high efficiency at frequencies up to 1 MHz. In this manner, all of the diode lasers may be made to pulse simultaneously to produce optical pulses at the optical coupler as short as 1 μs. In another embodiment, a laser diode module comprises 37 individually mounted diode lasers optically coupled to the fiber cable 120. The diode lasers are mounted such that they may receive voltage pulses simultaneously and with high efficiency at diverse frequencies including very high and very low frequencies. In this manner, all of the diode lasers may be made to pulse simultaneously or non-simultaneously to produce optical pulses at the optical coupler. Alternatively, the laser diode module 110 may comprise one diode laser optically coupled to a single optical fiber.

Delivery Device

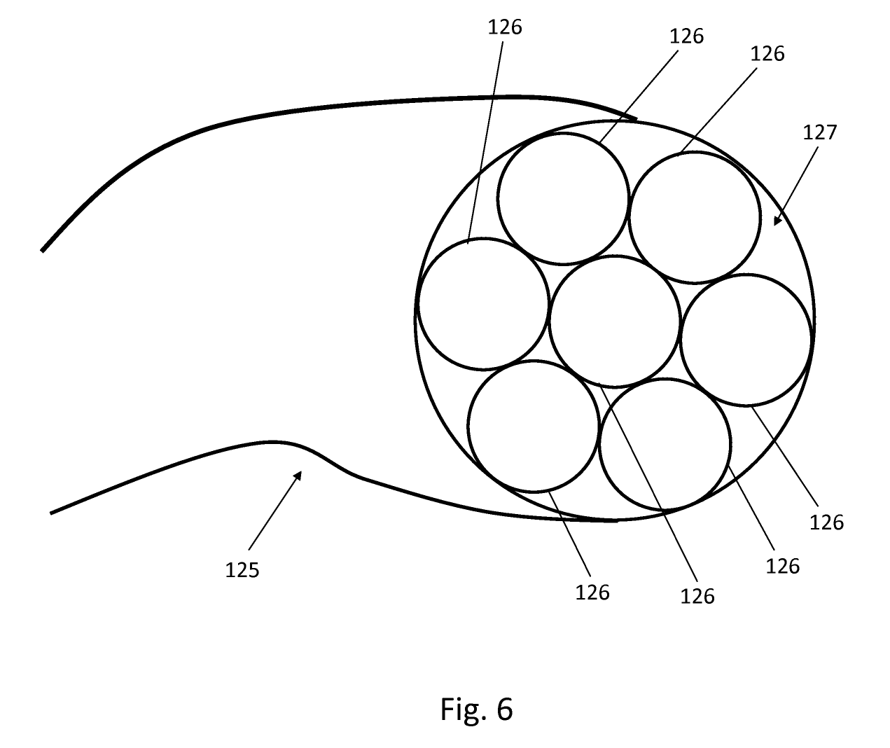

FIG. 6 illustrates delivery catheter 125 having a plurality of optical fibers 126 that reside within the light delivery channel 127 of the delivery catheter 125. The light delivery channel 127 is the portion of the delivery catheter 125 through which light may travel. In some embodiments, the light delivery channel 127 is essentially filled with one or more optical fibers 126. In other embodiments, the light delivery channel 127 is a lumen or void. In various embodiments, the light delivery channel 127 has a diameter greater than 0.35 mm and less than 0.65 mm. In other embodiments, the light delivery channel 127 has a diameter greater than 0.05 mm and less than 1.5 mm. In yet another embodiment, the light delivery channel 127 has a diameter of 0.5 mm. The delivery catheter 125 may comprise one or more optical fibers.

FIG. 6 depicts a delivery catheter 125 having a central circular optical fiber surrounding by six circular optical fibers of equal size. In another embodiment, the delivery catheter comprises a central circular optical fiber surrounded by six circular optical fibers of equal size, which are further surrounded by 12 circular optical fibers of equal size for a total of 19 circular optical fibers of equal size. Such a delivery catheter may include a separate laser diode optically coupled to each of the optical fibers of the delivery catheter.

In another embodiment, the delivery catheter has a single core 350 μm in diameter or 300 μm to 400 μm in diameter. In another embodiment the delivery catheter has a single core 500 μm in diameter or 400 μm to 500 μm in diameter. In another embodiment the delivery catheter has a single core 650 μm in diameter or 500 μm to 650 μm in diameter. In another embodiment the delivery catheter has a single core 700 μm in diameter or 650 μm to 750 μm in diameter. In another embodiment the delivery catheter has a single core 1000 μm in diameter or 750 μm to 1,200 μm in diameter.

In another embodiment, the delivery catheter 125 is a fiber cable 120 with the same number of cores as the fiber cable inside the chassis. In another embodiment, the delivery catheter is coiled to ensure mode filling. In many embodiments, the delivery catheter diameter is larger than the diameter of the fiber cable. In some embodiments, the delivery catheter 125 is a delivery fiber. In various embodiments, the delivery catheter is a hollow tube. Optical fibers 126 may be placed inside the delivery fiber or hollow tube.

In some embodiments, at least one fiber in the delivery catheter 125 can be used to deliver light from the target to a camera or an eyepiece. This can be used to aide placement of delivery catheter 125 near prostate. Such embodiments may be used on their own without a cytoscope or any other device. In some embodiments, delivery catheter 125 can be inserted directly into the body.

The delivery catheter may be passed into an cystoscope for viewing and targeting tissue. The cystoscope may include an optical filter so that the laser emission does not damage viewing systems. The optical filter may be configured to transmit light emitted by the aim diode if an aim diode is present.

In one embodiment, the delivery catheter 125 is passed through the working channel 290 of the cystoscope 200 so as to direct light energy towards the interior of the prostate gland 220. The distal end of the delivery catheter 125 and the region where the laser beam hits the wall of the prostate may be visible through the cystoscope 200. In another embodiment, the delivery catheter 125 can include a collimating lens to provide irradiance that varies slowly with fiber-tissue separation.

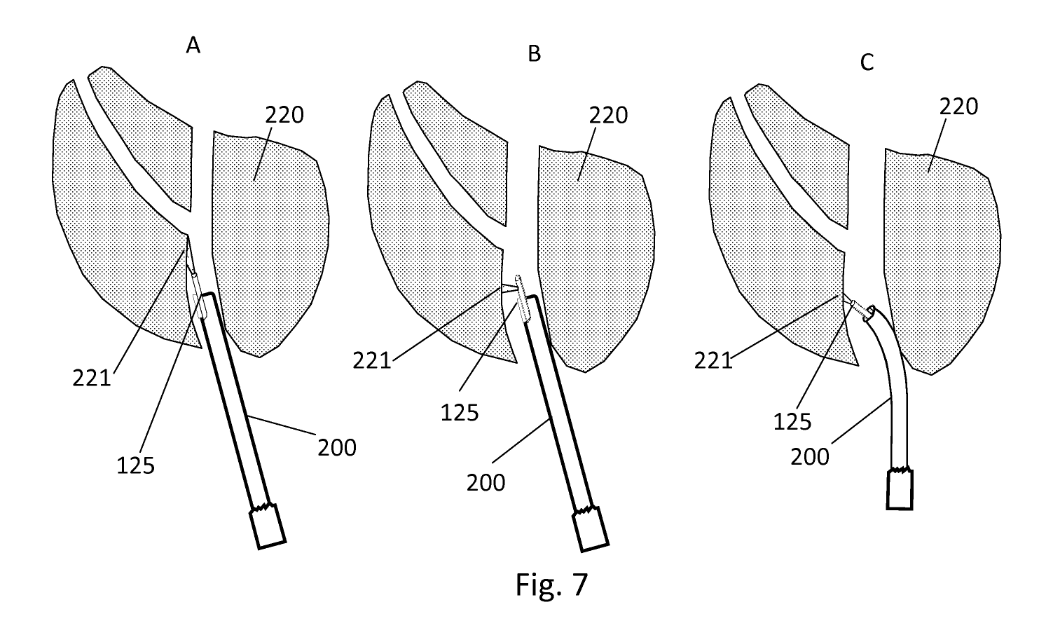

In another embodiment, as shown in FIG. 7B, the distal end of the delivery catheter reflects light at a 70-90 degree angle from the axis of the fiber towards the target prostatic tissue 221, and may optionally include a collimating lens to ensure the irradiance treating tissue is constant. The delivery catheter 125 may comprise a telescoping lens at the distal end to vary the irradiance at the treatment site.

As shown in FIG. 7C, the distal end of the delivery catheter 125 may curve or bend along with the distal end of the cystoscope 200. The distal end face of the delivery catheter 125, which may be a mono-mode or multi-mode filament, can be positioned so as to direct the light energy transmitted there through toward the wall of the prostate to treat an abnormal tissue, such as BPH, as shown for example in FIG. 7C. The delivery catheter 125 may be directed at a target prostatic tissue 221, thereby creating an ablation of the abnormal tissue and a coagulation of the surrounding tissue and/or underlying tissue. Coagulation of this tissue at the treatment site is desirable as it may result in hemostasis, which may reduce bleeding at the target treatment region.

In an exemplary embodiment, the cystoscope 200 is flexible and the delivery catheter 125 can be curved while articulating the end of the cystoscope 200. Generally, the delivery catheter 125 is flexible such that the curve radius of the distal end of the cystoscope 200 determines substantially the curve of the distal end of the delivery catheter. In one embodiment, cystoscope 200 is replaced with a second catheter and at least a portion of the delivery catheter 125 is inside the second catheter.

In an exemplary embodiment, the system 100 parameters can be adjusted such that the fluence at the tissue is 9 kW/cm², the pulse duration is 120 ms, and the pulse repetition rate is 8 Hz. In other embodiments, the pulse duration is between 80 ms and 160 ms; and the pulse repetition rate is between 4 Hz and 12 Hz. In yet another embodiment, the pulse duration is between 10 ms and 800 ms while the pulse repetition rate is between 1 Hz and 60 Hz. The laser diode pulse parameters can be adjusted such that the tissue damage due to coagulation is limited to a depth of 500 μm below the ablated surface.

In one method of treating prostatic tissue, successive layers of prostatic tissue are removed by sweeping the distal end of the tissue treatment system back and forth. In one example of sweeping back and forth, the distal end is moved right, then left, then right, and then left. This pattern of sweeping back and forth may be repeated until enough layers have been removed.

Coupler

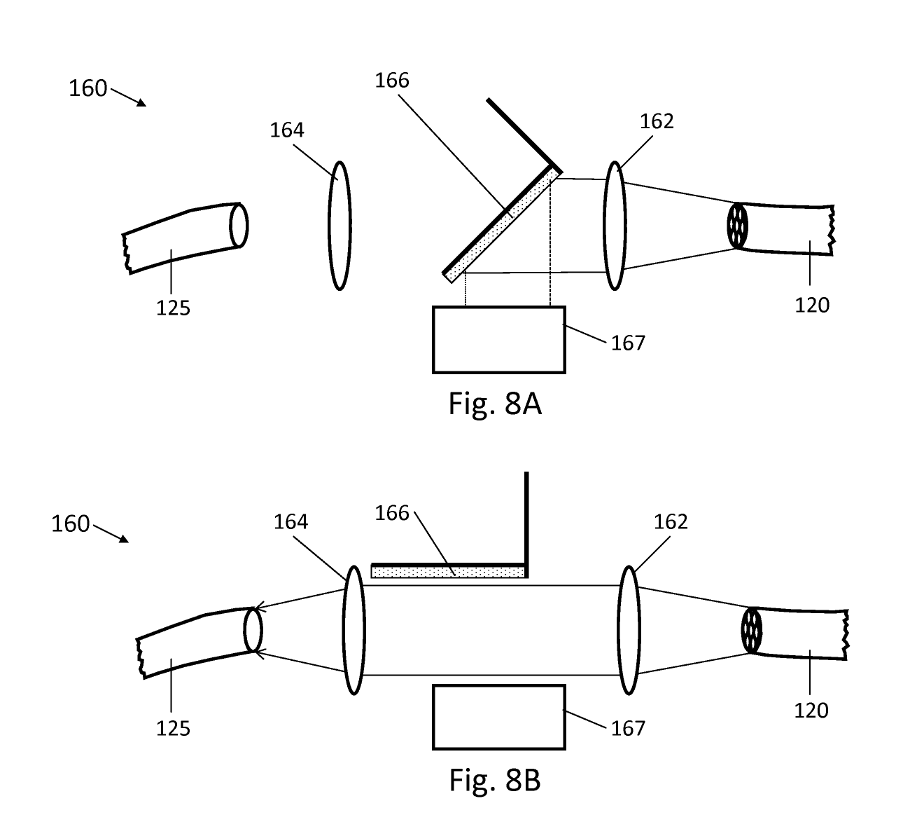

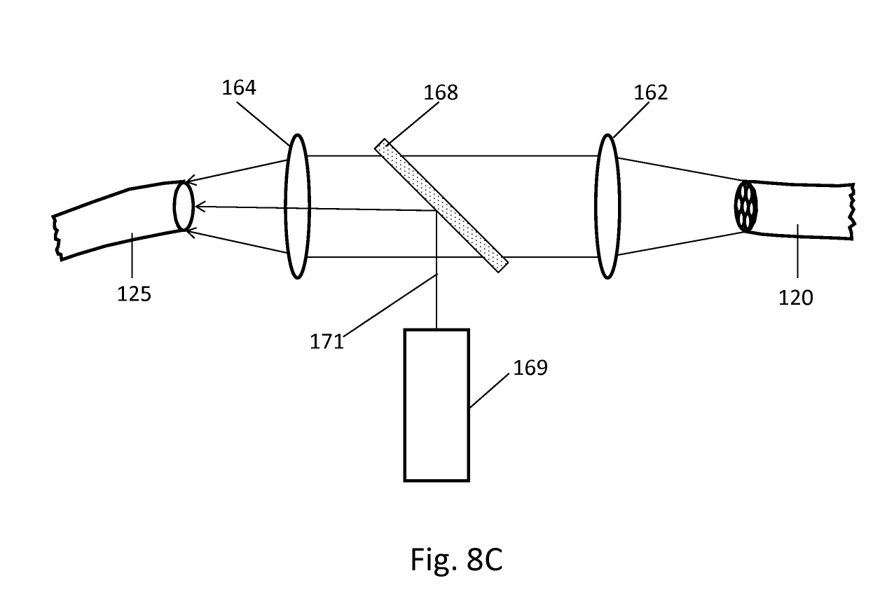

In an exemplary embodiment, the system 100 includes a coupler 160 that optically couples a distal end of fiber cable 120 to a proximal end of delivery catheter 125. Coupler 160 may further include a means to homogenize the beams emitted from the fiber cable 120. Coupler 160 may include a collimation lens 162 and a coupling lens 164, as shown for example in FIG. 8A. In various embodiments, collimation lens 162 has a numerical aperture greater than or equal to the numerical aperture of the fiber cable 120, while coupling lens 164 has a numerical aperture greater than or equal to the numerical aperture of the delivery catheter 125. Coupling lens 164 couples the light transmitted there through and projects the light energy into the proximal end face of the delivery catheter 125. This embodiment of coupler 160 is particularly useful for transmitting light energy from a multi-mode fiber to a mono mode fiber, as shown in FIG. 8A.

In some embodiments, collimation lens 162 and coupling lens 164 are spaced sufficiently apart to allow for insertion of a shutter mirror 166 and a beam-dump 167. The shutter mirror 166 is movable between an ON position and an OFF position, as shown for example in FIGS. 8A and 8B. In the OFF position (as shown in FIG. 8A), the shutter mirror 166 deflects the light energy transmitted through the collimation lens 162 into the beam dump 167. In the ON position (as shown in FIG. 8B), the shutter mirror 166 allows light energy to be transmitted from the collimation lens 162 to the coupling lens 164 to facilitate delivery of light energy through the delivery catheter 125 during treatment. Moving the shutter mirror 166 between the ON and OFF position allows a user to stop and start transmission of light energy during treatment without having to activate and de-activate the laser diode module 110. The insertion of a shutter mirror 166 and beam-dump 167 increases the treatment safety with system 100 since it allows the laser light energy to shut off completely and considerably faster than stopping the current to the laser diode module 110. Shutting the light energy off completely with shutter mirror 166 is advantageous since shutting off the light energy by changing the diode current or voltage can lead to unwanted pulses of light or very long spontaneous emission tails. Thus, shutting off the light energy “completely” with the shutter mirror 166 stops light energy from escaping , thereby preventing inadvertent treatment with light energy and improving safety during the procedure. The shutter mirror 166 and beam-dump 167 combination also allows for increased long-term reliability, since steady state laser operation is sometimes preferable over turning the laser diode module 110 on and off during treatment. Shutter mirror 166 is typically solenoid driven to be in the ON or OFF position. The mirror is typically driven to be either in the ON or OFF position rather than left between positions. Shutter mirror 166 allows the light energy to be deflected while preventing a shutter motor that moves the mirror from overheating due to the deflected light energy. Beam-dump 167 is a thick-walled metal box with an aperture or hole to allow the deflected light energy to enter the box, thereby preventing the light energy from overheating other components of system 100. In another embodiment, the beam dump comprises a light escape channel that directs the light to a heat sink located remotely from the delivery catheter. In another embodiment, the beam dump comprises a light escape channel that directs the light to a heat sink located remotely from the delivery catheter.

In another embodiment, coupler 160 may includes an aim diode 169, as shown for example in FIG. 8C. An aim diode 169 is typically a low-powered laser diode that emits a visible wavelength so that visible light 171 can be used to align or aim the distal end of the delivery catheter 125. In this embodiment, the aim diode 169 emits visible light 171, which is deflected by a mirror, such as dichroic mirror 168, through the coupling lens 164 and into the proximal end face of the delivery catheter 125. Typically, the light energy from the aim diode is transmitted near the center of the delivery catheter 125 to facilitate alignment of the distal end face of the delivery catheter 125 with the desired treatment area prior to and during the treatment process. The treatment area may be any tissue including but not limited to prostatic tissue. In one embodiment, the treatment area is inner prostatic tissue. The dichroic mirror 188 may be configured to pass light energy having a desired wavelength for use in the light energy ablation treatment (e.g., light from the laser diode module 110) while reflecting the wavelength of the visible light 171 emitted by aim diode 168. For example, the dichroic mirror 188 may allow light energy having a wavelength of 400-450 nm to pass through the mirror, while reflecting light energy transmitted by the aim diode 168, wherein the aim diode 168 light energy has a wavelength between 600 and 700 nm.

In embodiments where the laser diode module 110 generates significant waste heat that needs to be removed to prevent overheating, the laser diode module 110 may rest on a heat sink 112 to absorb the excess heat. The heat sink 112 may be cooled with a peltier cooler, refrigerated water, or blowing air.

Control Module

The control module 140 can control the operation of the system. The control module 140 may contain an embedded processor and logic circuits such as a field-programmable gate array (FPGA). The processor of the control module 140 may comprise a tangible medium having instructions of a computer readable program embodied thereon. The control module 140 may comprise programmable read-only-memory which further comprises the programs to implement algorithms which define system operation. The control module may comprise means to receive information in the form of voltage signals from the user interface 150, the footswitch 142, temperature monitors, the laser diode module 110, and a card reader for security card 144. In some embodiments, the control module 140 further comprises means to transmit information in the form of voltage signals to the DC power supply 170, the pulse generator 130, the laser diode module 110, and the user interface 150. The control module 140 may comprise a means to receive DC power or AC power. The control module 140 may also comprise the means to evaluate security codes transmitted from the security card 144.

Pulse Generator

The pulse generator 130 may comprise a frequency reference, for example a timer, and a means to generate voltage pulses. In one embodiment, the pulse generator 130 receives encoded instructions from the control module. The instructions contain information describing the desired amplitude of the voltage pulse, the duration of the voltage pulse, and the repetition frequency of the voltage pulse. In some embodiments, the pulse generator 130 comprises the means to generate voltage pulses at frequencies from 1 mHz to 1 MHz. The pulse generator 130 may comprise a means to generate pulses that are square, sinusoidal or saw-toothed. The pulse generator 130 may comprise a means to generate pulses with peak amplitudes of 1 V, 4.5 V, or 10 V. The pulse generator 130 may further comprise a means to transmit the voltage pulses to the laser diode module. In one embodiment, the pulse generator 130 comprises a means to transmit its status conditions to the control module.

User Interface

In some embodiments, the user interface 150 comprises a means to receive information from the user. In one embodiment, the user interface is a keyboard and a display. In another embodiment, the user interface 150 is a screen, such as a touch screen or a screen having a cursor controlled by direction keys or a mouse. The user interface 150 may further comprise a means for transmitting signals to the control module and a means for receiving signals from the control module. In one embodiment, the user interface 150 comprises a means for the user to select pulse duration, pulse energy, and pulse frequency. The user interface 150 may comprise a means to display system status information such as error conditions, usage rates, or laser emission.

DC Power Supply

The DC power supply 170 may comprise a means to receive AC power from outside the system. The DC power supply 170 may comprise a means to convert AC power to DC power. In one embodiment, the DC power supply 170 converts 110 V AC power to 24 V DC power. In another embodiment, the DC power supply 170 provides multiple DC voltages, wherein the multiple DC voltages power various system modules. In yet another embodiment, the power supply 170 is an AC power supply rather than a DC power supply.

Although the concepts disclosed herein have been described in connection with many different embodiments of the present invention, those of ordinary skill in the art will understand that many other modifications can be made to the disclosed embodiments without departing from the present invention. Accordingly, the above description should not limit the scope of the present invention. Instead, the scope of the present invention should be determined entirely by reference to the claims that follow.

What is claimed is:

1. A prostatic treatment system comprising:
a laser that emits light with a wavelength between 390 nm and 430 nm; and
a light delivery channel comprising a proximal end coupled with the laser, and a distal end, wherein the light delivery channel delivers light from the laser to the distal end.

2. The prostatic treatment system of claim 1, further comprising a cystoscope having a working channel, the light delivery channel having a portion that is located inside the working channel such that the prostatic treatment system transmits light from the laser through the cystoscope.

3. The prostatic treatment system of claim 1, wherein the laser emits light with a wavelength between 400 nm and 410 nm.

4. The prostatic treatment system of claim 1, wherein the laser emits light with a wavelength between 415 nm and 425 nm.

5. The prostatic treatment system of claim 1, wherein the prostatic treatment system has a maximum power output between 6 W and 60 W.

6. The prostatic treatment system of claim 1, wherein the prostatic treatment system vaporizes prostatic tissue with a power output between 15 W and 25 W.

7. The prostatic treatment system of claim 1, wherein the light exiting the distal end of the light delivery channel has a spot diameter between 0.5 mm and 2.5 mm.

8. The prostatic treatment system of claim 1, wherein the light delivery channel comprises one or more optical fibers.

9. The prostatic treatment system of claim 1, wherein the light delivery channel has a diameter greater than 0.35 mm and less than 0.65 mm.

10. The prostatic treatment system of claim 1, wherein the light delivery channel has a diameter greater than 0.05 mm and less than 1.5 mm.

11. The prostatic treatment system of claim 1, wherein the prostatic treatment system emits a flux greater than or equal to 1 kW/cm².

12. The prostatic treatment system of claim 1, wherein the prostatic treatment system emits a flux less than or equal to 10 kW/cm².

13. A prostatic treatment system that treats prostatic tissue having oxygenated hemoglobin, the system comprising:
a laser diode module that emits light near an absorption peak of the oxygenated hemoglobin; and
a light delivery channel having a proximal end and a distal end, the proximal end is optically coupled with the diode laser module, the light delivery channel delivers light from the laser to the distal end, and the light from the distal end vaporizes the prostatic tissue.

14. The prostatic treatment system of claim 13, wherein the laser diode module emits the light with a wavelength between 400 nm and 425 nm.

15. The prostatic treatment system of claim 13, wherein the prostatic treatment system emits the light from the distal end with a maximum flux of at least 1 kW/cm² and vaporizes the prostatic tissue by emitting a fluence of less than 5 kW/cm².

16. The prostatic treatment system of claim 13, wherein the prostatic treatment system has a maximum power output greater than 6 W and less than 30 W.

17. A method of treating target prostatic tissue, comprising:
inserting a light delivery channel having a proximal end and a distal end through a urethra, wherein the proximal end is optically coupled with a laser;
positioning the distal end of the light delivery channel near the target prostatic tissue; and
pulsing the laser with light having a wavelength between 390 nm and 430 nm.

18. The method of claim 17, wherein the pulsing the laser comprises emitting a light flux energy within a range from 500 W/cm² to 5,000 W/cm² to the target prostatic tissue.

19. The method of claim 17, wherein the pulsing the laser comprises emitting a fluence of less than 10 kW/cm².

20. The method of claim 17, wherein the prostatic treatment system has a maximum power output between 6 W and 60 W.

21. The method of claim 17, wherein the pulsing the laser comprises pulsing the laser with light having a wavelength between 400 nm and 410 nm.

22. The method of claim 17, wherein the pulsing the laser comprises pulsing the laser with light having a wavelength between 415 nm and 425 nm.
